(19) United States
(12) Patent Application Publication
(10) Pub. No.: US 2017/0027876 A1
(43) Pub. Date: Feb. 2, 2017

(54) PROTEIN-BASED ENTERIC COATING FOR ORAL DOSAGE FORMS

(71) Applicant: 9286-3620 QUEBEC INC., Levis (CA)

(72) Inventor: Romain Caillard, Quebec (CA)

(73) Assignee: 9286-3620 QUEBEC INC., Levis, QC (CA)

(21) Appl. No.: 15/106,485

(22) PCT Filed: Dec. 19, 2014
(86) PCT No.: PCT/CA2014/051241

§ 371 Date: Jun. 20, 2016

Related U.S. Application Data
(60) Provisional application No. 61/919,336, filed on Dec. 20, 2013.

(51) Int. Cl. A61K 9/48; A61K 31/525; A23P 10/30; A61K 35/747; A23P 20/10; A61K 9/00; A61K 36/28

(52) U.S. Cl. CPC A61K 9/4891; A61K 9/0053; A61K 9/4866; A61K 31/525; A61K 36/28; A61K 35/747; A23P 20/10; A23P 10/30

(57) ABSTRACT

The use of chemically modified proteins as enteric or gastroresistant coatings for oral dosage forms containing an active ingredient is described. Typical enteric coatings are made from synthetic polymers and function by acting as a barrier to gastric medium penetration. In some cases, the manufacture of such coatings requires the use of hazardous chemicals and the safety of some of the synthetic polymers that have been used in enteric coatings have been questioned. Protein-based enteric coatings of the present description present a more natural alternative to enteric coatings produced from synthetic polymers to provide gastroresistance to oral dosage forms. Protein-based film-forming solutions and uses thereof in coating oral dosage forms are also provided.

### CROSS-REFERENCE TO RELATED APPLICATIONS

This is the U.S. National Stage of International Application No. PCT/CA2014/051241, filed Dec. 19, 2014, which was published in English under PCT Article 21(2), which in turn claims the benefit of U.S. Application No. 61/919,336, filed Dec. 20, 2013.

### FIELD

This present description relates to the use of protein-based materials as enteric coatings for oral dosage forms. More particularly, the present description relates to oral dosage forms enterically coated with chemically modified proteins.

### BACKGROUND

Film coating is widely used in the formulation of oral dosage forms (e.g., capsules, softgels, tablets, etc.) for various reasons, such as protecting active pharmaceutical ingredients from light exposure, moisture, oxygen, pH/enzyme-induced degradation, and/or masking unpleasant taste (Abu Diak et al., 2007; Li et al., 2010). Film coatings used to modify oral dosage forms are usually referred to as extended-release coatings (time release coatings or sustained release coatings) or delayed-release coatings (enteric coatings or gastroresistant coatings).

In general, enteric coatings function by acting as a barrier to gastric medium penetration (Cunningham & Fegely, 2001; Dias et al., 2010; Meghal et al., 2011). More particularly, they present a surface that is stable and relatively impermeable to the highly acidic and protease-rich environment of the stomach, but breaks down rapidly in a less acidic environment such as in the small intestine. Accordingly, materials suitable for such enteric coatings are generally acid- and protease-resistant (i.e., non-proteinaceous) polymers that exhibit low erosion and low swelling when in gastric medium, leading to low penetration of the gastric medium into the coating layer. At neutral pH, however, the polymers start to swell and erode, leading to dissolution of the oral dosage form and release of the active ingredient.

Enteric coatings are generally made from synthetic polymers such as Polyvinyl Acetate Phthalate (PVAP), Poly Methacrylic Acid-Ethyl Acrylate (PMA-EA), Poly Methacrylic Acid-Methyl Methacrylate (PMA-MM), Cellulose Acetate Phthalate (CAP), Cellulose Acetate Trimellitate (CAT), Hydroxy Propryl Methyl Cellulose Phthalate (HPMCP), or Hydroxy Propryl Methyl Cellulose Acetate Succinate (HPMCPAS) (Porter et al, 2009). However, the use of some of these polymers and derivatives thereof has been restricted in many countries for a variety of reasons (e.g., health concerns, government regulations, public pressure). For instance, the use of phthalate derivatives has been restricted in Europe and in the US since 1999. Moreover, in some cases, the manufacture of some of these polymers involves the use of hazardous chemical agents. For instance, methylation of cellulosic materials is generally achieved using chloromethane, a toxic and flammable compound. Furthermore, the clean-up of equipment (e.g., pan coater) following the use of synthetic polymers can be a long process (e.g., several hours), generally requiring an alkali treatment combined with mechanical action (e.g. brushing, high pressure spray-cleaning) prior to rinsing with deionized water. In some cases, solvents may also be needed.

Enteric coatings are generally prepared by applying a film-forming solution onto the surface of an oral dosage form (e.g., capsules, softgels, tablets, granules, pellets, etc.). The film-forming solution usually contains the acid-resistant polymer, as well as other compounds such as plasticizer(s), colorant(s) or other additives (e.g., anti-adhesive agents, surfactants, flavoring agents, etc.) (Porter et al., 2009).

Enteric coatings are traditionally applied to oral dosage forms using a pan coating system. This method involves spraying the film-forming solution/suspension on the surface of the dosage forms, while the dosage forms are kept in continuous movement in a rotating pan. This technique requires maintaining a controlled balance between spray and evaporation, by applying heated air flow in contact with the surface of the oral dosage form. The process is continued until the desired amount of coating is applied (the desired thickness) (Porter et al., 2009), and the coating weight gain per unit can be monitored throughout the process.

Techniques other than pan coating exist in order to deposit a polymer film on oral dosage forms. These other methods include Wurster coating (air suspension coating) and dip coating.

The preparation of an enteric coating can be a long process (e.g., several hours) and, in some cases, a sub-coating step may be required. The sub-coating step is generally used for coating capsules in order to seal them, or to avoid an interaction between film polymer and drug (e.g., interaction between an enteric film containing phthalyl groups and tablets containing an alkaline drug) (Porter et al., 2009).

There is therefore a need for new enteric coatings. There is also a need for enteric coatings that can be manufactured without the use of hazardous materials, that are more natural, and/or that can be applied and cleaned in a more time and/or cost efficient manner.

### SUMMARY

In some aspects, the present description relates to an enterically coated oral dosage form comprising a core comprising at least one active ingredient, wherein the core is enterically coated with a chemically modified protein.

In some embodiments, the chemically modified protein may comprise a chemical modification that causes a decrease in isoelectric point (pi) below that of the corresponding unmodified protein. In more specific embodiments, the decrease in pI may be a decrease of 0.1, 0.2, 0.3, 0.4, 0.5, 0.6, 0.7, 0.8, 0.9, 1, 1.5, 2, 2.5, or 3 units, below that of the corresponding unmodified protein.

In some embodiments, the chemically modified protein may comprise a chemical modification that causes a decrease in solubility at acidic pH as compared to that of the corresponding unmodified protein.

In some embodiments, the chemically modified protein may comprise a chemical modification that increases the ability of the protein to resist degradation by pepsin when in film-form, as compared to that of the corresponding unmodified protein.

In some embodiments, the chemically modified protein may be chemically modified by succinylation, octenyl-succinylation, methylation, acetylation, phosphorylation, acylation, sulfation, carboxylation, arylation, alkylation, deamidation, glutarylation, reaction with an anhydride, ethylenediaminetetraacetic dianhydride (EDTAD), reaction with fluorescein isothiocyanate, reaction with dimethylaminonaphtylsulfonyl chloride (dansyl chloride), or any combination thereof. In some embodiments, the chemically modified protein may be chemically modified by succinylation. In some embodiments, the chemically modified protein may be chemically modified at least at 1%, 5%, 10%, 15%, 20%, 25%, 30%, 35%, 40%, 45%, 50%, 55%, 60%, 65%, 70%, or 75% of its available sites. In some embodiments, the chemically modified protein may be chemically modified at between 1% and 100%, 10% and 100%, or 20% and 100% of its available sites.

In some embodiments, the chemically modified protein may be a chemically modified water soluble protein. In some embodiments, the chemically modified protein may comprise at least 1%, at least 1.5%, at least 2%, at least 2.5%, at least 3%, at least 3.5%, at least 4%, at least 4.5%, at least 5%, at least 6%, at least 7%, at least 8%, at least 9%, or at least 10% of lysine residues. In some embodiments, the chemically modified protein may comprise between 1% and 20%, 1% and 15%, 1% and 10%, 1.5% and 20%, 1.5% and 15%, or 1.5% and 10% of lysine residues. In some embodiments, the chemically modified protein may comprise at least 3%, at least 4%, at least 5%, at least 6%, at least 7%, or at least 8% of glutamate and aspartate residues.

In some embodiments, the chemically modified protein may comprise between 3% and 20%, 4% and 20%, 5% and 20%, 6% and 20%, 7% and 20%, or 8% and 20% of glutamate and aspartate residues.

In some embodiments, the chemically modified protein may be from animal or plant source. In some embodiments, the chemically modified protein may be from a food protein. In some embodiments, the chemically modified protein may be from a dairy protein. In some embodiments, the chemically modified protein may be from a leguminous protein. In some embodiments, the chemically modified protein may be a chemically modified soy protein, whey protein, pea protein, nut protein, peanut protein, bean protein, lentil protein, wheat globulin protein, corn globulin protein, rice globulin protein, sunflower globulin protein, rape globulin protein, casein protein, animal albumin protein, egg ovalbumin protein, animal collagen protein, or any combination thereof. In some embodiments, the chemically modified protein may be a chemically modified soy protein or whey protein. In some embodiments, the soy protein is from soybean protein extract (SPE) or soybean protein isolate (SPI). In some embodiments, the whey protein is from whey protein extract (WPE), whey protein concentrate (WPC), or whey protein isolate (WPI).

In some embodiments, the enteric weight gain of the oral dosage form is about 2% to about 30% w/w, about 3% to about 20% w/w; about 5% to about 20% w/w; about 5% to about 18% w/w, or about 8% to about 15% w/w, relative to the initial weight of the oral dosage form.

In some embodiments, the enteric coating may exhibit swelling of at least 30%, 40%, 50%, 60%, 70%, 80%, 90%, 100%, 110%, or 120% by weight, relative to the initial weight of the enteric coating, within 15 minutes upon incubation in simulated gastric fluid (SGF). In some embodiments, less than 20%, less than 15%, less than 10%, or less than 5% of the active ingredient may be released, relative to the initial weight of the active ingredient, upon incubation for 1 hour in simulated gastric fluid (SGF). In some embodiments, at least 20%, 25%, 30%, 35%, 40%, 45%, 50%, 55%, 60%, 35%, 70%, 75%, 80%, 85%, 90%, or 95% of the active ingredient, relative to the initial weight of the active ingredient, may be released upon subsequent incubation for 2 hours in simulated intestinal fluid (SIF).

In some embodiments, the enteric coating may further comprise a plasticizer. In some embodiments, the plasticizer may be a polyhydric alcohol; an acetate ester; a phthalate ester; a glyceride; an oil; or any combination thereof. In some embodiments, (a) the polyhydric alcohol is glycerol, propylene glycol, or a combination thereof; (b) the acetate ester is glyceryl triacetate, triethyl citrate, or a combination thereof; (c) the phthalate ester is diethyl phthalate; (d) the glyceride is an acetylated monoglyceride; or (e) the oil is castor oil, mineral oil, or a combination thereof.

In some embodiments, the oral dosage form may further comprise a sub-coating between the enteric coating and the core. In some embodiments, the sub-coating may comprise: a cellulosic based coating, a vinyl based coating, a glycol, an acrylic coating, a carbohydrate-based coating. In some embodiments, the sub-coating may comprise: (a) a cellulosic based coating which is: hydroxypropylmethylcellulose, hydroxypropylcellulose, hydroxyethylcellulose, cellulose acetate, or any combination thereof; (b) a vinyl based coatings which is: poly vinyl pyrrolidone, poly vinyl alcohol, poly vinyl pyrrolidone, a poly vinyl acetate copolymer, poly vinyl alcohol, a poly ethylene glycol copolymer, or any combination thereof; (c) a glycol which is poly ethylene glycol; (d) an acrylic coating which is an amino alkyl methacrylate copolymer; (e) a carbohydrate-based coating which is: maltodextrins, polydextrose, or a combination thereof; or (f) any combination of (a) to (e).

In some embodiments, the oral dosage form may take the form of an enterically coated tablet, capsule, caplet, softgel, lozenge, pellet, or granule. In some embodiments, the active ingredient may be a drug, a vitamin, a nutritional supplement, a probiotic, or any combination thereof. In some embodiments, the active ingredient may be: an antiagreggant, an antiangiogenic, an antiarrythmia, an antibiotic, an antidepressor, an antifungal, an antiviral, an anticholinergic, an antiepileptic, an anticoagulant, an anticonvulsive, an antidiarrhea, an antihistaminic, an antihypertensive, an antiinflammatory, an analgesic, an antalgics, an antipsychotic, an antispasmodic, a synthetic antithyroid, an anxiolytic, a beta-blocker, a cardiotonic, a diuretic, a hypnotic, a hypoglycemic, a hypolipemic, an inhibitor of conversion enzyme, an inhibitor of angiotensin II, an interferon, a mucolytic, a nootropic, a phenylethylamine, a sartan, a triptan, a vitamin, a flavonoid, a peptide, a hormone, an enzyme, a prebiotic, a probiotic, a mineral, an ellagic acid, an omega-3 fatty acid, an omega-6 fatty acid, a terpene, or any combination thereof.

In some aspects, the present description relates to a film-forming solution comprising: (a) a chemically modified protein as defined herein; (b) a plasticizer as defined herein; and (c) a solvent. In some embodiments, the solution may comprise about 1%, 2%, 3%, 4%, 5%, 6%, 7%, 8%, 9%, or 10% to about 14%, 15%, 16%, 17%, 18%, 19%, or 20% w/w of the chemically modified protein. In some embodiments, the solution may comprise about 0.02% to 30% w/w of the plasticizer. In some embodiments, the solvent may be water and/or a mixture of an alcohol and water. In some embodiments, the alcohol may be ethanol, methanol, or isopropanol. In some embodiments, the solvent may be water.

In some embodiments, the film-forming solution as defined herein may be used for enterically coating an oral dosage form comprising at least one active ingredient (e.g., as defined herein).

In some aspects, the present description relates to the use of the film-forming solution as defined herein for enterically coating an oral dosage form comprising at least one active ingredient.

In some aspects, the present description relates to a method for enterically coating an oral dosage form, the method comprising coating the oral dosage form with the film-forming solution as defined in any one of claims 36 to 41, and drying the enterically coated oral dosage form.

In some aspects, the present description relates to an enterically coated oral dosage form prepared by a method as defined herein.

In some aspects, the present description relates to an enterically coated oral dosage form comprising at least one active ingredient wherein the dosage form is enterically coated with a chemically modified soy protein or a chemically modified whey protein. In some embodiments, the soy protein or the whey protein may be succinylated at between 20% and 100% of its available sites. In some embodiments, the enteric weight gain of the dosage form may be about 2 to about 30% w/w.

In some aspects, the present description relates to a solution comprising: a chemically modified soy protein or a chemically modified whey protein; a plasticizer; and a solvent. In some embodiments, the soy protein or the whey protein may be succinylated at between 20% and 100% of its available site. In some embodiments, the solution may comprise: about 1 to 20% (w/v) of a chemically modified soy protein or a chemically modified whey protein; about 0.02 to 30% (w/v) of a plasticizer; and a solvent. In some embodiments, the plasticizer may be polyhydric alcohols (e.g. glycerol, propylene glycol), acetate esters (e.g. glyceryl triacetate, triethyl citrate), phthalate esters (e.g. diethyl phthalate), glycerides (e.g. acetylated monoglycerides) or oils (e.g. castor oil, mineral oil). In some embodiments, the solvent may be water and/or alcohol.

In some aspects, the present description relates to the use of a solution for enterically coating an oral dosage form comprising at least one active ingredient.

In some aspects, the present description relates to a method for enterically coating an oral dosage form comprising coating the oral dosage form with a solution as defined in any one of claims 4 to 8 and drying the enterically coated oral dosage form.

### BRIEF DESCRIPTION OF THE DRAWINGS

In the appended drawings:

FIG. 1 shows the relationship between polymer concentration and viscosity of chemically modified protein-based film-forming solutions as compared to a standard Eudragit™ solution. WPI: Whey Protein Isolate succinylated at 40%; WPC: Whey Protein Concentrate succinylated at 50%; SPI: soy protein isolate succinylated at 100%.

FIG. 2 shows typical drying kinetics of succinylated protein-based film-forming solutions as compared to a standard Eudragit™ solution. WPI: 40%-succinylated WPI containing 14% protein w/w; WPC: 50%-succinylated WPC containing 14% protein w/w; SPI: 100%-succinylated SPI containing 14% protein w/w; or Eudragit: Eudragit™ L30 D-55 film containing 17.4% w/w and 0.9% triethyl citrate w/w.

FIGS. 3A-3D and 4a-4D show the typical effects of protein concentration (FIGS. 3A-3D) and the typical effects of plasticizer/protein ratio (FIG. 4A-4D) on the mechanical properties of protein-based films. Results obtained from films prepared from 50%-succinylated WPC are shown in FIG. 3A-3D. Results obtained from films prepared from 40%-succinylated WPI using glycerol as the plasticizer are shown in FIG. 4A-4D. Four parameters were measured: (A) The film's maximum force at break; (B) the film's elongation at break; (C) penetration work (related to the film's “endurance”); and (D) the film's Young's modulus (elastic modulus).

FIG. 5 shows a typical comparison between the mechanical properties of the protein-based films as compared to a standard Eudragit™ film. The protein-based films shown in FIG. 5 contain 10% w/w protein and 4% glycerol (0.4 glycerol/protein ratio). WPC: film prepared from WPC succinylated at 50%; WPI: film prepared from WPI succinylated at 40%; and SPI: film prepared from SPI succinylated at 100%.

FIG. 6 shows typical erosion kinetic profiles from protein-based films in simulated gastric fluid (SGF) as a function of time. The protein-based films shown in FIG. 6 contain 14% w/w protein and a glycerol/protein ratio of 0.35. “WPC”: 50%-succinylated WPC; “WPI”: 50%-succinylated WPI; and “SPI”: 50%-succinylated SPI.

FIG. 7 shows typical swelling kinetics from protein-based films in simulated gastric fluid as a function of time, as compared to a standard Eudragit™ film. The protein-based films shown in FIG. 7 contain 12% w/w protein and a glycerol/protein ratio of 0.35. “WPI”: 50%-succinylated WPI; and “SPI”: 50%-succinylated SPI.

FIG. 8 shows typical release profiles of oral dosage forms enterically coated with protein-based films using USP disintegration apparatus USP<701>, in which the oral dosage forms were exposed to simulated gastric fluid (SGF) from 0 to 60 min, and then to simulated intestinal fluid (SIF) from 60 to 180 min. Release is expressed as % of the total amount of active ingredients initially contained in the oral dosage forms.

### DESCRIPTION OF ILLUSTRATIVE EMBODIMENTS

The present description generally relates to the use of chemically modified proteins as enteric or gastroresistant coatings for oral dosage forms.

### Chemical Modifications

In some aspects, proteins suitable for enteric coatings of the present description are chemically modified. As used herein, the expression “chemically modified protein” refers to a suitable protein of the present description that has been covalently modified using a reagent (e.g., a water-soluble reagent), such that the chemical modification: (i) causes a decrease in the isoelectric point (pi) of the protein below that of the corresponding unmodified (e.g., native) protein; (ii) causes a decrease in solubility of the protein at acidic pH as compared to that of the corresponding unmodified (e.g., native) protein; (iii) increases the ability of the protein to resist degradation by gastric proteases (e.g., pepsin) when in film form or when enterically coating an oral dosage form, as compared to that of the corresponding unmodified (e.g., native) protein; or (iv) any combination of (i) to (iii). Without being bound by theory, such chemical modifications are believed to increase the ability of suitable proteins of the present description to provide gastroresistance to oral dosage forms enterically coated therewith, yet provide an enteric coating that enables release of an active ingredient in a higher pH environment (e.g., in the intestine).

In some embodiments, chemically modified proteins of the present description have a modified isoelectric point (pi) that is between 0.1 and 3 units lower than the pl of the corresponding unmodified (e.g., native) protein. In some embodiments, chemically modified proteins of the present description have a modified pl that is 0.1, 0.2, 0.3, 0.4, 0.5, 0.6, 0.7, 0.8, 0.9, 1, 1.5, 2, 2.5, or 3 units lower than the pl of the corresponding unmodified (e.g., native) protein. Of course, the actual value of the pl of the chemically modified protein depends on the pl of the corresponding unmodified (e.g., native) protein. In particular embodiments, chemically modified proteins (e.g., soy or whey proteins) of the present description may have a modified pl below 5.5, below 5.0, or below 4.5. In more particular embodiments, chemically modified proteins (e.g., soy or whey proteins) of the present description have a modified pl between 4.4 and 3.5, more particularly between 3.9 and 3.6, and still more particularly between 3.8 and 3.7.

In some embodiments, suitable chemical modifications may include succinylation (Subirade et al., 1992; Schwenke et al.,1992), octenyl-succinylation, acetylation (Gruener and lsmond, 1997), methylation, acylation, phosphorylation, sulfation, carboxylation, arylation, alkylation, deamidation, glutarylation (Singh et al., 1995), reaction with an anhydride, ethylenediaminetetraacetic dianhydride (EDTAD) (Hwang D-C., Damodaran S., 1996), reaction with fluorescein isothiocyanate, reaction with dimethylaminonaphtylsulfonyl chloride (dansyl chloride), or any combination thereof. In some embodiments, chemical modifications can be carried out as shown in WO/2009/149553.

In some embodiments, suitable proteins of the present description are chemically modified at one or more of their available sites. As used herein, the expression “available site” refers to one or more types of functional groups present in suitable proteins of the present description (e.g., in particular amino acid side chains) which are amendable to chemical modification by a reagent in accordance with the present description. The skilled person would understand that the identity of the available site depends on the choice of reagent and/or the choice of chemical modification. For example, succinylation is a chemical reaction in which a succinyl group (—CO—CH₂—CH₂—CO—) is added, for example, to an amino group (e.g., the epsilon-amino group of a lysine residue) by a nucleophilic attack. Addition of a succinyl group to a lysine's epsilon-amino group changes lysine's charge from +1 to −1. In such a case, the available site is the epsilon-amino groups of lysine residues.

In some embodiments, chemical modifications of the proteins of the present description are performed by reacting the proteins with an anhydride. For example, proteins may be succinylated using succinic anhydride wherein mainly lysyl residues are modified. In some aspects, the proteins are firstly re-hydrated (e.g., for a few hours) and succinic anhydride is then added to the protein solution continuously. During modification, pH is maintained between 8-8.2 using an alkali. At the end of modification, modified proteins are dried (e.g., using spray-drying or lyophilization).

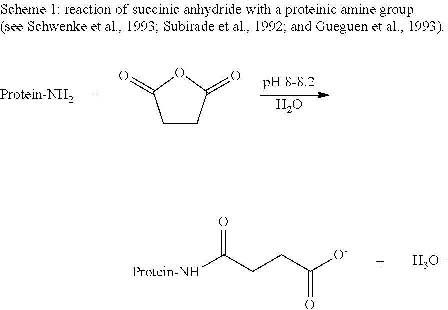

In some embodiments, protein succinylation can be carried out as shown in WO/2009/149553.

Some chemical modifications including succinylation, methylation, acetylation, phosphorylation, glutarylation, acylation and others occur in nature as post-translational modifications to proteins. As such, proteins of the present description which are chemically modified by such processes may be considered as being more natural and/or safer, as compared to synthetic polymers used in many enteric coatings that are employed currently. Accordingly, in some embodiments, the protein-based coatings of the present description may be used in the replacement of plastic-based enteric coatings.

In some embodiments, proteins of the present description may be chemically modified to various extents/degrees (e.g., between 1% and 100%) of their available sites, for example as shown in WO/2009/149553. In some embodiments, the extent/degree of chemical modification that is necessary for the protein-based enteric coatings of the present description can depend on a number of factors including: the type of chemical modification or reagent that is selected (e.g., succinylation, octenyl-succinylation, acetylation, acylation, methylation, etc.); the type of protein that is to be modified (e.g., soy protein, whey protein, rice protein, etc.); the presence of other agents such as plasticizers; or the desired physical/mechanical properties of the enteric coating (e.g., pI, acid solubility, maximum force at break, elongation at break, penetration work, Young's modulus, coating/film weight, erosion properties, swelling properties, active ingredient release properties) or film-forming solution (e.g., viscosity). Selecting the extent/degree of the chemical modification in view of the above factors would be within the scope of the skilled person, in view of the present disclosure.

In some embodiments, the chemically modified protein is sufficiently modified to provide gastroresistance to oral dosage forms enterically coated therewith, yet provide an enteric coating that enables release of an active ingredient in a higher pH environment (e.g., in the intestine). In some embodiments, the chemically modified protein is chemically modified at least at 1%, 5%, 10%, 15%, 20%, 25%, 30%, 35%, 40%, 45%, 50%, 55%, 60%, 65%, 70%, or 75% of its available sites. In some embodiments, the chemically modified protein is chemically modified at between 1% and 100%, 10% and 100%, or 20% and 100% of its available sites. In some embodiments, the percentage of modification may be determined experimentally using an amino blocker such as ortho-phthadialdehyde, tri-benzene sulfonic acid, or ninhydrin. In some embodiments, the degree of chemical modification may be controlled by calculating the ratio of reactives (e.g., concentration of protein; initial available site (e.g., NH₂) content; concentration of chemical modification reagent), for example as described in WO/2009/149553. In view of the present disclosure, the skilled person would be able to determine the suitable degree of chemical modification depending on the type of protein used, in accordance with the present description.

### Proteins

In some aspects, proteins suitable for the present description are those amenable for chemical modification as described herein such that the chemically modified proteins: (i) have a decreased isoelectric point (pi) below that of the corresponding unmodified (e.g., native) protein; (ii) have a decreased solubility at acidic pH as compared to that of the corresponding unmodified (e.g., native) protein; (iii) have an increased ability to resist degradation by gastric proteases (e.g., pepsin) when in film form or when enterically coating an oral dosage form, as compared to that of the corresponding unmodified (e.g., native) protein; or (iv) any combination of (i) to (iii).

The term “protein” or “proteins” as used herein means that the material originates from proteinic material either from animal or plant origin (vegetable and cereals), but may be native or in an altered state such as, but not limited to, pre-treated, denatured and/or hydrolyzed. Heterogeneous mixtures of different types of proteins are encompassed by the terms “protein” or “proteins”. In some embodiments, proteins of the present description may include globular proteins and/or food proteins. In some embodiments, proteins of the present description are from natural sources (i.e., not recombinantly produced).

In some embodiments, suitable proteins of the present description should be water soluble or at least partially water soluble. As used herein, the expression “water soluble protein” refers to proteins which are sufficiently soluble in water or in an aqueous solution to enable them to be chemically modified (e.g., succinylated) in accordance with the present description.

In some embodiments, suitable proteins of the present description have primary structures (i.e., amino acid compositions) which are favorable for chemical modification and/or for stability in low pH environments (e.g., comprise a relatively high proportion of acidic residues). In some embodiments, proteins suitable for the present description have at least 1%, 1.5%, 2%, 2.5%, 3%, 3.5%, 4%, 4.5%, 5%, 6%, 7%, 8%, 9%, or 10% lysine residues. In a particular embodiment, proteins suitable for the present description have at least 2% lysine residues. In some embodiments, proteins suitable for the present description have between 1% and 20%, 1% and 15%, 1% and 10%, 1.5% and 20%, 1.5% and 15%, or 1.5% and 10% of lysine residues. In some embodiments, proteins suitable for the present description have at least 5%, 6%, 7%, 8%, 9%, 10%, 11%, 12%, 13%, 14%, or 15% of acidic residues (i.e., glutamate and aspartate residues). In a particular embodiment, proteins suitable for the present description have at least 8% acidic residues (i.e., glutamate and aspartate residues).

In the present examples, soy and whey proteins were selected for chemical modification in part because of their relatively low cost and great availability. However, all proteins regardless of their source are polymers of amino acids and the skilled person would understand that the present description should not necessarily be limited proteins from soy and whey, but rather proteins which fulfill the criteria as described herein. In this regard, whey proteins (which include alpha- and beta-lactoglobulins) are soluble in water, have a lysine content of about 8.7%, and a total glutamate/aspartate content of about 14.5%. Soy proteins (and their main components vicilin and legumin) are soluble in water, have a lysine content of about 4.8%, and a total glutamate/aspartate content of about 14.2%. Proteins from other sources that fulfill the criteria described herein may also be suitable for use in the protein-based enteric coatings of the present description.

More particularly, pea proteins (and their main components vicilin and legumin) are soluble in water, have a lysine content of about 5.8%, and a total glutamate/aspartate content of about 15.5%. Bean proteins (and their main components vicilin and legumin) are soluble in water, have a lysine content of about 8.1%, and a total glutamate/aspartate content of about 15.5%. More generally, leguminous proteins such as soy proteins, pea proteins, nuts and peanuts proteins, bean proteins, lentil proteins, etc., and their main components may be eligible. Thus, the above mentioned proteins may be suitable.

Furthermore, wheat globulins are soluble in water, have a lysine content of about 2.2%, and a total glutamate/aspartate content of about 10.5%. Corn globulins are soluble in water, have a lysine content of about 2%, and a total glutamate/aspartate content acid of about 8.1%. Rice proteins and their globular components (albumin, globulin) are soluble in water, have a lysine content of about 3.4%, and a total glutamate/aspartate content of about 12.9%. Thus, the above mentioned proteins may be suitable. More generally, loaf globular proteins such as wheat globulins, corn globulins, rice globulins, etc., may also be suitable.

Sunflower globulins are soluble in water, have a lysine content of about 2%, and a total glutamate/aspartate content of about 9.5%. Rape globulins are soluble in water, have a lysine content of about 2.5%, and a total glutamate/aspartate content of about 8.8%. Thus, the above mentioned proteins may be suitable. More generally, oleaginous globular proteins (which include sunflower globulins, rape globulins, etc.) may also be suitable.

Caseins are partially soluble in water, have a lysine content of about 7.2%, and a total glutamate/aspartate content of about 11.5%. Animal albumins are soluble in water, have a lysine content of about 9.9%, and a total glutamate/aspartate content of about 15.5%. Egg ovalbumins are soluble in water, have a lysine content of about 5.2%, and a total glutamate/aspartate content of about 12.1%. Animal collagens are partially soluble in water, have a lysine content of about 4.2%, and a total glutamate/aspartate content of about 9.2%. Thus, the above mentioned proteins may be suitable.

Accordingly, oral dosage forms of the present description may be enterically coated with a chemically modified protein that is from animal or plant source (e.g., a food protein, dairy protein, or a leguminous protein). In some embodiments, the chemically modified protein is a chemically modified soy protein, whey protein, pea protein, nut protein, peanut protein, bean protein, lentil protein, wheat globulin protein, corn globulin protein, rice globulin protein, sunflower globulin protein, rape globulin protein, casein protein, animal albumin protein, egg ovalbumin protein, animal collagen protein, or any combination thereof. In particular embodiments, the chemically modified protein is chemically modified soy protein or whey protein. In specific embodiments, soy protein is from soybean protein extract (SPE) or soybean protein isolate (SPI). In specific embodiments, the whey protein is from whey protein extract (WPE), whey protein concentrate (WPC), or whey protein isolate (WPI).

In contrast, proteins which are insoluble in water may be difficult to chemically modify or may have amino acid compositions (e.g., high proportion of hydrophobic amino acids, low proportion of acidic amino acids, and/or low proportion of modifiable residues such as lysines) which make them unfavorable for use in the protein-based enteric coatings of the present description. For example, corn prolamin, namely zein, is insoluble in water, a has a lysine content of about 0.2%, and a total glutamate/aspartate content of about 0.7%. Rice prolamins are insoluble in water, have a lysine content of about 0.6%, and a total glutamate/aspartate content of about 1.8%. Wheat gliadins have low solubility in water, have a lysine content of about 0.3%, and a total glutamate/aspartate content of about 1.3%. Wheat glutenins have low solubility in water, have a lysine content of about 0.8%, and a total glutamate/aspartate about 2.4%. Such proteins may not be suitable. More generally, vegetal prolamins, vegetal gliadins and wheat glutenins may not be suitable.

In some embodiments, protein-based coatings of the present description may be considered as being more natural than traditional enteric coatings prepared using synthetic polymers, particularly when the type of chemical modifications used occur in nature as post-translational modifications to proteins (e.g., including succinylation, methylation, acetylation, phosphorylation, glutarylation, acylation). Accordingly, protein-based coatings of the present description, in part because of their “naturality”, may raise less safety concerns than traditional enteric coatings prepared using synthetic polymers.

### Film-Forming Solutions and Coatings

In some aspects, the chemically modified proteins of the present description are used to prepare a film-forming solution suitable for coating an oral dosage form. In some embodiments, the film-forming solution can be prepared by hydrating a protein suitable for the present description in a solvent such as water, or a mix of water and ethanol, to form a protein solution. In some embodiments, the protein concentration may range between 1-20% w/w. In some embodiments, film-forming solution may comprise about 1%, 2%, 3%, 4%, 5%, 6%, 7%, 8%, 9%, or 10% to about 14%, 15%, 16%, 17%, 18%, 19%, or 20% w/w of the chemically modified protein. A plasticizer or a mix of plasticizers can then be added to the protein solution. In some embodiments, the plasticizer can belong to any class of plasticizing agents: polyhydric alcohols (e.g., glycerol, propylene glycol), acetate esters (e.g., glyceryl triacetate, triethyl citrate), phthalate esters (e.g., diethyl phthalate), glycerides (e.g., acetylated monoglycerides) and oils (e.g., castor oil, mineral oil). In some embodiments, the concentration of the plasticizer(s) ranges between 0.02%-30% w/w. In some embodiments, other traditionally used additives can be added to the film-forming solution such as colorant(s), anti-adhesive agent(s), surfactant(s), flavoring agent(s), etc. The resulting film-forming solution can then be applied onto an oral dosage form (e.g., capsule, soft-gel, granule, tablet, pellet, etc.).

Accordingly, in some aspects, the present description relates to a film-forming solution comprising: (a) a chemically modified protein as defined herein; (b) a plasticizer; and (c) a solvent. In some embodiments, the film-forming solution comprises about 1% to 20% w/w of the chemically modified protein. In some embodiments, the solution comprises about 0.02% to 30% w/w of the plasticizer.

In some embodiments, the plasticizer is a polyhydric alcohol; an acetate ester; a phthalate ester; a glyceride; an oil; or any combination thereof. In further embodiments, the polyhydric alcohol is glycerol, propylene glycol, or a combination thereof; the acetate ester is glyceryl triacetate, triethyl citrate, or a combination thereof; the phthalate ester is diethyl phthalate; the glyceride is an acetylated monoglyceride; or the oil is castor oil, mineral oil, or a combination thereof. In some embodiments, the solvent comprises water. In other embodiments, the solvent is water or a mix of an alcohol (e.g., ethanol, methanol, and/or isopropanol) and water.

In some embodiments, film-forming solutions of the present description may be relatively easy to prepare, requiring only 30 min to 1 h, depending on the batch size. In comparison, a hypromellose acetate succinate suspension can require as much as 3 h of preparation before application onto oral forms. Furthermore, in some embodiments, protein-based coating solutions of the present description can be prepared using standard solids concentration and lead to low viscosity solutions. Moreover, in some embodiments, protein-based coatings of the present description demonstrate high adhesiveness and lead to high weight gain speeds, thus reducing coating time.

### Oral Dosage Forms

In some aspects, the film-forming solutions of the present description are used for enterically coating an oral dosage form. As used herein, the expressions “enteric coating” or “enterically coated” refers to a separate and distinct protein-based coating/layer that is applied to an oral dosage form containing an active ingredient, for example, in order to prevent an undesirable interaction and/or reaction between the active ingredient and the acidic environment of the stomach (gastroresistance). Examples of such undesirable interactions and/or reactions include premature release, premature degradation, and/or premature activation of the active ingredient upon administration, as well as interactions and/or reactions leading to undesirable effects to the subject resulting from having the active agent released in the stomach (e.g., stomach irritation, unpleasant release of gas/odors from the mouth). As used herein, the expressions “enteric coating” or “enterically coated” also refers to the ability of a protein-based coating of the present description to readily release the active ingredient in a higher pH environment (e.g., in the intestine).

As use herein, “protein-based coating” or “protein-based enteric coating” refers to coatings comprising chemically modified proteins of the present description in sufficient quantities such that the gastroresistant activity of the coating stems mainly from the presence of the chemically modified proteins. As used herein, the expression “protein-based” does not exclude the possibility of including other ingredients in the coating (e.g., plasticizers, additives, colorants, flavor enhancers, etc.).

Typical equipment used to coat oral dosage forms may be used to apply the film-forming solution. In some embodiments, the film-forming solution is applied using a pan coating system. Pan coating is usually performed using appropriate temperatures (inlet/outlet), spray rate or pan speed, as for any enteric coating application.

In some embodiments, coating equipment used with the protein-based film-forming solutions of the present description may be cleaned relatively quickly using only deionized water without mechanical action (e.g., brushing, high pressure spray-cleaning). This is in contrast to traditional plastics-based coatings, which generally require the use of an alkali treatment (and possibly other solvents) combined with mechanical action, prior to rinsing with deionized water—a cleaning process that can take several hours.

Once the coating process is completed, enterically coated oral dosage forms of the present description can be tested following the United States Pharmacopoeia (USP) Guidelines for delayed release devices. In some embodiments, enterically coated oral dosage forms of the present description meet the criteria defined in USP <701> or USP <724>.

In USP <701>, enterically coated oral dosage forms are tested using a disintegration apparatus. In this test, one oral dosage form is placed in each of the six tubes of the basket. After one hour of incubation in a simulated gastric fluid (SGF), no evidence of disintegration, cracking, or softening should be observed on the surface of the coated oral form and consequently, no release of the active ingredient should occur. Oral dosage forms should then disintegrate into simulated intestinal fluid (SIF) following the time specified in its monograph.

In USP <724>, oral dosage forms are tested using a dissolution apparatus. Twenty-four units are tested during two hours of incubation in gastric simulated conditions. Delayed release is respected if, at the end of experiments, the average release of the 24 tablets is not more than 10% dissolved and no individual unit is greater than 25% dissolved. Dissolution time in the intestine should then respect the monograph of the product.

As used herein, “simulated gastric fluid” or “SGF” refers to a solution that can be prepared by dissolving 2 g of sodium chloride, 7 mL of 37% hydrochloric acid, 1000 mL of HPLC grade water (pH 1.2) and pepsin (3.2 g, 924 units/mg of protein) as defined in US pharmacopoeia (The United States Pharmacopoeial Convention, Tests Solutions, USP Convention Inc., Rockville, Md., 2004, pp. 9-23).

As used herein, “simulated intestinal fluid” or “SIF” refers to a solution that can be prepared by combining 6.8 g of monobasic potassium phosphate dissolved in 250 mL of H PLC grade water, 190 mL of 0.2 N sodium hydroxide and 400 mL of HPLC grade water, adjusting the pH to 7.5±0.1 using 0.2 N sodium hydroxide, and bringing the final volume to 1000 mL with double-distilled water. Pancreatin is then added (10 g, activity equivalent to USP specification), as defined in US pharmacopoeia (The United States Pharmacopoeial Convention, Tests Solutions, USP Convention Inc., Rockville, Md., 2004, pp. 9-23).

In some aspects, the present description relates to an enterically coated oral dosage form comprising a core comprising at least one active ingredient, wherein the core is enterically coated with a chemically modified protein. In some embodiments, the enteric weight gain of the oral dosage form is about 2% to about 30% w/w, about 3% to about 20% w/w; about 5% to about 20% w/w; about 5% to about 18% w/w, or about 8% to about 15% w/w, relative to the initial weight of the oral dosage form (i.e., prior to coating with the protein-based coating of the present description).

In some embodiments, the enteric coating of the present description comprises a plasticizer as defined above. In some embodiments, the enteric coating of the present description comprises an additive. In some embodiments, the enteric coating of the present description comprises only ingredients that are generally recognized as safe (GRAS).

In some embodiments, the enterically coated oral dosage form of the present description further comprising a sub-coating between the enteric coating and the core. In some embodiments, the sub-coating may be useful for improving the adherence of the protein-based enteric coating of the present description to the surface or the oral dosage form. In some embodiments, sub-coatings include but are not limited to: cellulosic based coatings (hydroxypropylmethylcellulose, hydroxypropylcellulose, hydroxyethylcellulose, cellulose acetate), vinyl based coatings (poly vinyl pyrrolidone, poly vinyl alcohol, poly vinyl pyrrolidone, poly vinyl acetate copolymers, poly vinyl alcohol, poly ethylene glycol copolymers), glycols (poly ethylene glycol), acrylics (amino alkyl methacrylate copolymers), or other carbohydrates (maltodextrins, polydextrose).

In some embodiments, the enteric coatings of the present description exhibit swelling of at least 30%, 40%, 50%, 60%, 70%, 80%, 90%, 100%, 110%, or 120% by weight, relative to the initial weight of the enteric coating, within 15 minutes upon incubation in simulated gastric fluid (SGF).

In some embodiments, less than 20%, less than 15%, less than 10%, or less than 5% by weight, relative to the initial weight of the active ingredient, is released upon incubation for 1 hour in simulated gastric fluid (SGF). In some embodiments, at least 20%, 25%, 30%, 35%, 40%, 45%, 50%, 55%, 60%, 65%, 70%, 75%, 80%, 85%, 90%, or 95% of the active ingredient is released, relative to the initial weight of the active ingredient, upon subsequent incubation for 2 hours in simulated intestinal fluid (SIF). As used herein, release profile (e.g., % release of an active ingredient over time) relates to a ratio or percentage of the amount of an active ingredient that is released from an oral dosage form as compared to the total amount of the active ingredient that was initially contained in the oral dosage form, after a specified incubation time (e.g., minutes or hours). In some embodiments, the release profile can be expressed as Mt/Minf, which corresponds to the ratio of the amount of the active ingredient that is released at a time “t” (Mt) divided by the amount of the active ingredient released after an infinite amount of time “inf” (i.e., the total quantity of active ingredient in the oral dosage form; Minf). In some embodiments, the percentage release can be in terms of weight, mole, viable count, or using other units, depending on the type of the active ingredient being measured. For example, in the case of probiotics, active ingredient amounts of usually expressed as viable counts.

In some embodiments, oral dosage forms of the present description can take the form of an enterically coated tablet, capsule, caplet, softgel, lozenge, pellet, or granule.

### Active Ingredients and Uses

In some embodiments, oral dosage forms of the present description comprise a core comprising an active ingredient which is a drug (including veterinary drugs), a vitamin, a nutritional/dietary supplement, a probiotic, or any combination thereof. In some embodiments, oral dosage forms of the present description are for use in animals. In more particular embodiments, oral dosage forms of the present description are for use in a human, fish, farm animal or pets (such as cats, dogs, horses, pigs, and most monogastric mammals). In a more particular embodiment, oral dosage forms of the present description are for use in humans.

In some embodiments, the above mentioned active ingredients may include pharmaceutical active agents such as drugs, biological active agent (e.g., an enzyme or bacteria), nutraceutical agents such as vitamins. Non limiting examples include: an antiagreggant, an antiangiogenic, an antiarrythmia, an antibiotic, an antidepressor, an antifungal, an antiviral, an anticholinergic, an antiepileptic, an anticoagulant, an anticonvulsive, an antidiarrhea, an antihistaminic, an antihypertensive, an antiinflammatory, an analgesic, an antalgics, an antipsychotic, an antispasmodic, a synthetic antithyroid, an anxiolytic, a beta-blocker, a cardiotonic, a diuretic, a hypnotic, a hypoglycemic, a hypolipemic, an inhibitor of conversion enzyme, an inhibitor of angiotensin II, an interferon, a mucolytic, a nootropic, a phenylethylamine, a sartan, a triptan, a vitamin, a flavonoid, a peptide, a hormone, an enzyme, a prebiotic, a probiotic, a mineral, an ellagic acid, an omega-3 fatty acid, an omega-6 fatty acid, a terpene, or any combination thereof.

In some embodiments, the enteric coating of the present description lacks the active ingredient, which is present only in the core of the oral dosage form. In some embodiments, the core of oral dosage forms of the present description lacks the chemically modified proteins of the present description.

In some embodiments, enteric coatings of the present description can be used to protect an active ingredient from premature release, premature degradation, and/or premature activation in the mouth, esophagus, or stomach, and/or to promote release of the active ingredient in the intestine. In some embodiments, enteric coatings of the present description can be used to protect a subject being administered the oral dosage form from any potentially irritating effects that the active ingredient may have on the stomach. In some embodiments, enteric coatings of the present description can be used to reduce or avoid other undesirable effects that the active ingredient may have on a subject. For example, enteric coatings have been used in fish oil (omega-3 fatty acids) supplements to prevent the fish oil capsules from being digested in the stomach, which has been known to cause a fishy reflux (fish burps).

The present description will be more readily understood by referring to the following examples. The examples are illustrative of the wide range of applicability of the present description and are not intended to limit its scope. Modifications and variations can be made therein without departing from the spirit and scope of the invention. Although any methods and materials similar or equivalent to those described herein can be used in the practice for testing of the present description, the following methods and materials are described. The issued patents, published patent applications, and references that are cited herein are hereby incorporated by reference to the same extent as if each was specifically and individually indicated to be incorporated by reference. In the case of inconsistencies, the present disclosure will prevail.

### EXAMPLES

### Example A

### Material and Methods

Materials, Chemical modification of proteins, Erosion and Swelling experiments, Dissolution experiments, Dissolution data analysis, and Statistical analyses were carried out as described in PCT application No. PCT/CA2009/000819 (publication No. WO 2009/149553), unless otherwise mentioned herein.

### Example B

### Viscosity of Protein-Based Film-Forming Solutions vs. Eudragit™ Solution

Film-forming solution viscosity is a parameter to consider during coating development. Viscosity of the film-forming solution may have an impact on droplet size distribution, coating efficiency and film surface. Generally, film-forming solution having viscosities below 350 cP are easier to work with (Porter et al., 2009).

Film-forming solutions containing different concentrations of: (1) Whey Protein Isolate (WPI) succinylated at 40%; (2) Whey Protein Concentrate (WPC) succinylated at 50%; (3) soy protein isolate (SPI) succinylated at 100%; or (4) Eudragit™ L30 D-55 were prepared as follows: Water/solvent were firstly mixed with plasticizer(s), if present; Polymer materials were then progressively added; and Solutions/suspensions were gently stirred for 30-60 min at room temperature. Viscosities of the solutions were measured using a Brookfield viscometer.

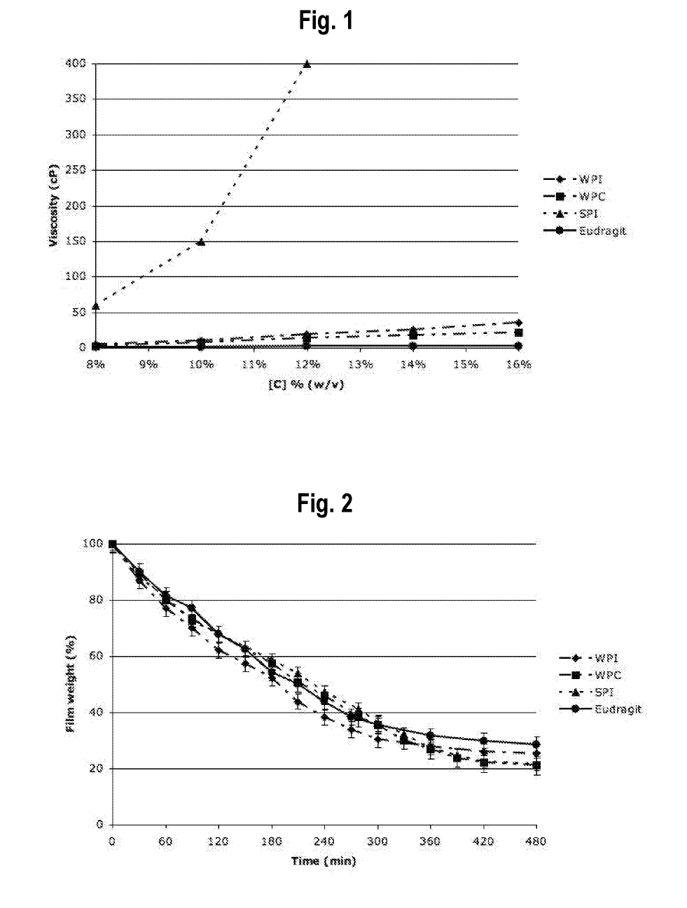

FIG. 1 illustrates the effect of polymer concentration (w/w) on film-forming solution viscosity. Film-forming solutions containing WPI succinylated at 40% and WPC succinylated at 50%, showed a low viscosity remaining below 40 cP even at a 16% w/w concentration. In contrast, a film-forming solution containing SPI succinylated at 100% showed a higher viscosity (greater than 350 cP for concentrations greater than 12%). Without being bound by theory, this difference in film-forming solution viscosity between succinylated WPI/WPC and succinylated SPI may be related to the higher succinylation rate of SPI and higher molecular weight, which both lead to higher protein hydrodynamic radii.

Viscosity of the film-forming solutions was noted to also increase with time (data not shown). “Older” solutions showed higher viscosities than freshly prepared film-forming solutions. This rheopectic behavior was particularly true for SPI and WPI, and, without being bound by theory, may be related to aggregation phenomena.

No effect of plasticizer type or plasticizer/protein ratio was noted (data not shown). Plasticizers tested included glycerol, propylene glycol, PEG 400, PEG 2000, and triethyl citrate. The plasticizer/protein ratios tested were between 0.1 and 2. In some embodiments, adding ethanol to the film-forming solutions containing higher concentrations of protein slightly decreased their viscosity (data not shown).

Regardless of the polymer concentration, all protein-based film-forming solutions tested showed higher viscosity values than Eudragit™ solution (2-4 cP).

### Example C

### Typical Drying Kinetics for Protein-Based Film-Forming Solutions

Film-forming solutions containing: (1) 40%-succinylated WPI containing 14% protein w/w; (2) 50%-succinylated WPC containing 14% protein w/w; (3) 100%-succinylated SPI containing 14% protein w/w; or (4) Eudragit™ L30 D-55 film containing 17.4% w/w and 0.9% triethyl citrate w/w were prepared as described in Example B. The film-forming solutions were then spread into Petri dishes and placed in an oven at 50° C. with relative humidity (RH) at about 55%. These temperature and RH conditions are comparable to those typically used during industrial pan coating of capsules/tablets. Drying kinetics were then followed by performing gravimetric measurements and the results are shown in FIG. 2, which presents relative film weight as a function of time (minutes) until stabilization.

Other film-forming solutions that were tested included those containing WPI succinylated at 50%, 75% and 100%; WPC succinylated at 50%; and SPI succinylated at 100%. The concentrations of protein tested included between 6% and 14%. Plasticizers tested included glycerol, propylene glycol, PEG 400, PEG 2000, and triethyl citrate. The plasticizer/protein ratios tested were between 0.1 and 2.

For all tested film-forming solutions, all of the drying kinetics showed the same general “shape”. The film weights of all the tested solutions tended to be stabilized at the same time, indicating no strong differences between the tested polymers in terms of their “drying aptitude”. The final film weight of each of the tested solutions depended on the solids content in each film-forming solution. Eudragit™ films showed higher final weight than the protein-based films.

No effect of plasticizer type or plasticizer/protein ratio was noted (data not shown). In some embodiments, including ethanol in the film-forming solutions increased the solution's drying speed in a concentration-dependent manner (data not shown).

### Example D

### Effects of Protein Type and Concentration on Mechanical Properties of Protein-Based Films

The effects of protein type and protein concentration on mechanical properties of protein-based films were analyzed. Film-forming solutions containing: (1) 40%-succinylated WPI containing 10, 12, 14 or 16% protein w/w; (2) 50%-succinylated WPC containing 10, 12, 14 or 16% protein w/w; or (3) 100%-succinylated SPI containing 10, 12, 14 or 16% protein w/w were prepared as described in Example B. Ethanol concentration for each film-forming solution was fixed at 20% (w/w). The plasticizer/protein ratio of the films was 0.4, and glycerol was used as plasticizer.

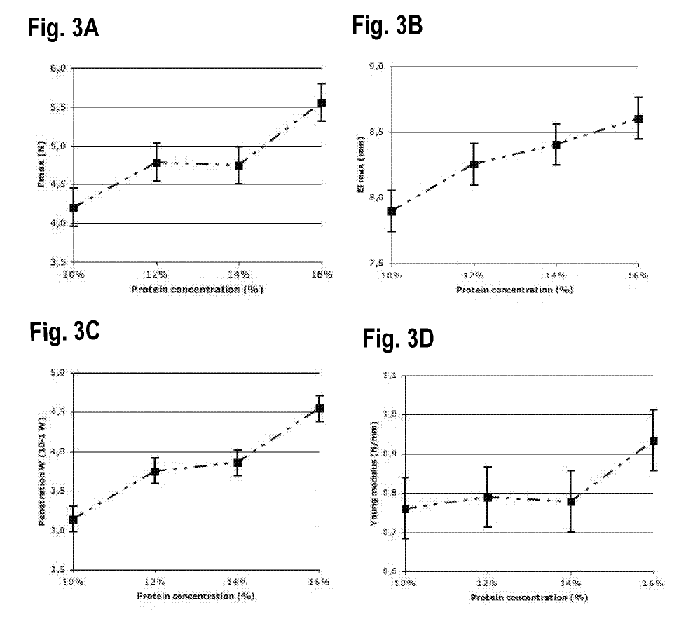

Mechanical properties were studied on 24 h old films using a penetrometer (TAXT-2). For each film, including Eudragit™ film, four parameters were measured: (a) The film's maximum force at break; (b) the film's elongation at break; (c) penetration work (related to the film's “endurance”); and (d) the film's Young's modulus (elastic modulus). Typical results are shown in FIGS. 3A-3D with respect to films prepared from 50%-succinylated WPC. However, regardless of the type of protein used to prepare the film, the protein concentration influenced the films' mechanical properties in the same manner. Increasing the protein concentration in each of the film-forming solutions led to an increase in all mechanical parameters. This resulted in stronger and more elastic films.

### Example E

### Effects of Plasticizer Type and Plasticizer/Protein Ratio on Mechanical Properties of Protein-Based Films

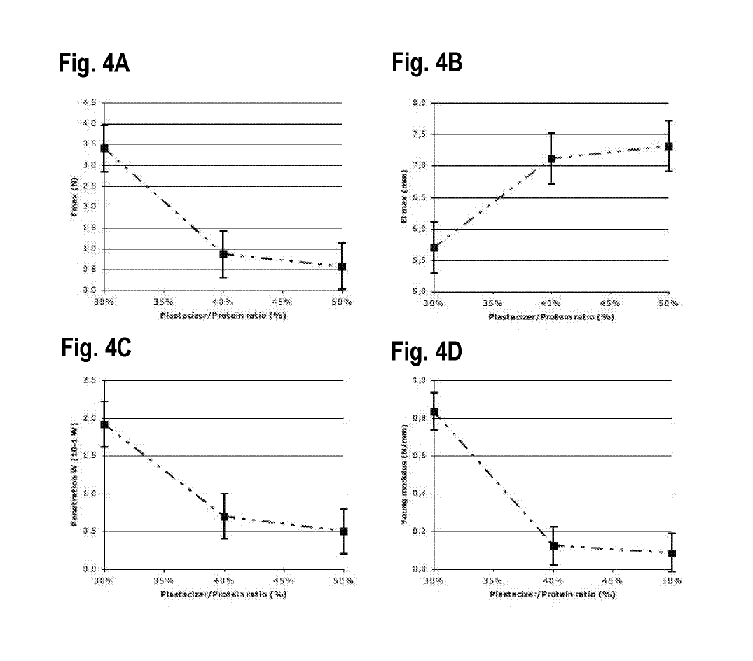

The effects of plasticizer type and plasticizer/protein ratio on mechanical properties of protein-based films were analyzed. To this effect, film-forming solutions containing: (1) 40%-succinylated WPI; (2) 50%-succinylated WPC; or (3) 100%-succinylated SPI were prepared having different plasticizer/protein ratios, as described in Example A. Typical results are shown in FIG. 4A-4D with respect to films prepared from 40%-succinylated WPI using glycerol as the plasticizer at plasticizer/protein ratios of between 0.3 (30%) and 0.5 (50%). Other plasticizers tested include polyethylene glycol 400 (PEG-400), polyethylene glycol 2000 (PEG-2000), and propylene glycol (PG).

Experiments revealed that, regardless of the type of protein used to prepare the film (i.e., WPI succinylated at 40%, WPC succinylated at 50%, or SPI succinylated at 100%), an increase in plasticizer concentration led to a decrease of the films' maximum force at break, mechanical modulus and penetration work. An increase in plasticizer concentration also generally led to an increase in films' elongation at break. In other words, an increase in plasticizer concentration led to a decrease of films' strength and an increase of its flexibility.

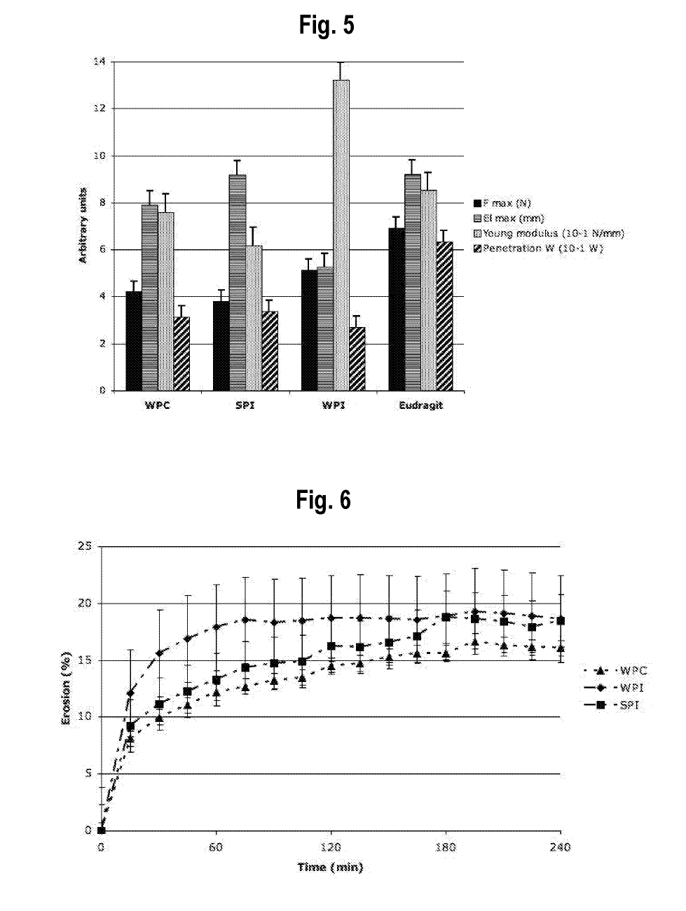

Comparisons between the mechanical properties of the protein-based films and a standard Eudragit™ film were performed. FIG. 5 shows a typical comparison between the mechanical properties of the protein-based films containing 10% w/w protein and containing 4% glycerol (0.4 glycerol/protein ratio). “WPC”: film prepared from WPC succinylated at 50%, “WPI”: film prepared from WPI succinylated at 40%, and “SPI”: film prepared from SPI succinylated at 100%. The protein-based films displayed comparable mechanical properties to a standard Eudragit™ film.

### Example F

### Erosion Kinetics from Protein-Based Films in Simulated Gastric Fluid

The erosion kinetic profiles of protein-based films were analyzed. Films were prepared using a casting method as described above in Examples C to E. Erosion experiments were performed directly on those films. Proteins that were tested include: WPC succinylated at 50%; WPI succinylated at 50%; and SPI succinylated at 100%, prepared as described in Example B. Protein concentrations tested were 10-14%. Plasticizers tested included PEG 400, glycerol, and PG. Plasticizer/protein ratios were between 0.25 (25%) and 0.5 (50%).

Films erosion was measured by conducting dissolution experiments in a USP II apparatus (USP <711>, 2008). Films were placed in a simulated gastric fluid (SGF) and absorbance at 280 nm was measured as a function of time, using a spectrophotometer. Typical erosion kinetics profiles from protein-based films in simulated gastric fluid as a function of time are shown in FIG. 6. In this figure, “WPC”: 50%-succinylated WPC; “WPI”: 50%-succinylated WPI; and “SPI”: 50%-succinylated SPI, the protein concentrations were at 14%, and the plasticizer/protein ratio was at 0.35 (35%) using glycerol as plasticizer. Erosion reached 10% to 15% after 30 min of experiment and final erosion was approximately 20% for all tested films.

No effect of plasticizer concentration, plasticizer type or protein concentration was noted for all tested films (data not shown). All films showed fast erosion in SGF starting at the first minutes of experiment.

### Example G

### Swelling Kinetics from Protein Based Films in Simulated Gastric Fluid

The swelling kinetic profiles of protein-based films were analyzed. Films were prepared for testing as described above in Examples C to E and F. Proteins that were tested include: WPC succinylated at 50%; WPI succinylated at 50%; and SPI succinylated at 100%, prepared as described in Example B. Protein concentrations tested were 10-14%. Plasticizers tested included PEG 400, glycerol, and PG. Plasticizer/protein ratios were between 0.3 and 0.5 (30% and 50%).

The swelling of films was measured by conducting dissolution experiments in a USP II apparatus (USP <711>, 2008). Swelling kinetics were followed by measuring films weight intake as a function of time.

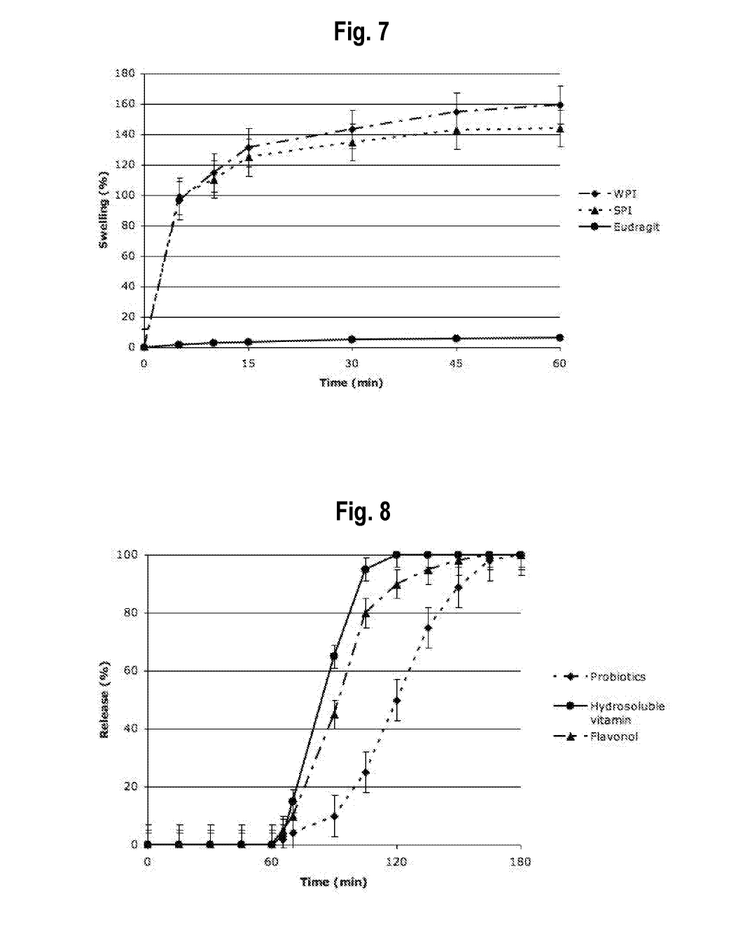

Typical swelling kinetics from protein-based films in simulated gastric fluid as a function of time versus a standard Eudragit™ film, is shown in FIG. 7. In this figure, “WPI”: 50%-succinylated WP; and “SPI”: 50%-succinylated SPI, the protein concentrations were at 12% w/w, and the plasticizer/protein ratio was at 0.35 (35%) using glycerol as plasticizer.

No effect of plasticizer concentration, plasticizer type or protein concentration was noted for all tested films (data not shown). All films showed fast swelling in SGF starting at the first minutes of experiment. After only 5 min of experiment, films swelling rate was 100%. After 1h of experiment, swelling reached 160% for all films. In other words, after 60 min of experiment, a 1 g film absorbed about 1.6 g of gastric fluid, containing pepsin (the stomach main protease). In comparison, after 1 h of experiment, Eudragit™ films showed a swelling rate of 6% (27 times lower). Protein based films showed a gastric swelling 4 to 120 times higher than traditional enteric coatings after incubation times 24 times lower.

### Example H

### Release Profiles of Oral Dosage Forms Enterically Coated with Protein-Based Films

The release profiles of capsules (capsule “0”) containing the following active ingredients were tested:
100 mg of riboflavin (vitamin B2) and 300 mg of microcrystalline cellulose as a filler (“hydrosoluble vitamin” in FIG. 8);
250 mg of a goldenrod extract and 200 mg of microcrystalline cellulose as a filler (“flavonol” in FIG. 8); or
250 mg of a Lactobacillus casei lyophilisate and 200 mg of microcrystalline cellulose as a filler (“probiotics” in FIG. 8).

Prior to application of protein-based coatings, capsules were first sealed using a hydroxypropyl cellulose (HPC)-based sub-coating (weight gain of about 4%). HPC was applied using a pan coater prior to enteric coating. HPC was applied in order to ensure sealing of capsules.

Protein-based film-forming solutions were prepared as described in Example B. Solutions were applied onto capsule “0” using a pan coater using the coating parameters as described in Table A below.

TABLE A

| | | |
|---|---|---|
| Inlet air temperature: | 75-95° | C. |
| Product temperature: | 35-45° | C. |
| Pan drum speed: | 16-20 | rpm |
| Spray rate: | 20-30 | mL · min⁻¹ |
| Distance between nozzle/product: | 10-14 | cm |

| | |
|---|---|
| Spray angle: | 10-90° |

FIG. 8 illustrates typical release profiles obtained from capsules coated with succinylated proteins using a disintegration apparatus USP<701>, as described below for Examples 1-15. The results in FIG. 8 show the release profiles of oral dosage forms that were coated using 50%-succinylated WPI containing 35% of glycerol (expressed in plasticizer/protein ratio in w/w). Enteric weigh gain of each oral dosage form was about 15% w/w. Riboflavin release and golden rod extract release were followed by measuring absorbance at 445 nm and 317 nm, respectively, as a function of time. Release is expressed as the % of total amount of active ingredient released as a function of time. Probiotic release was followed by measuring viable counts as a function of time. Briefly, aliquots were taken from vessels and serially diluted if necessary. 1 mL of each dilution was then plated in MRS agar and incubated 48 h at 37° C. Release results are expressed as the % of the total viable bacteria released as a function of time.

Of note, as shown in FIG. 8, no release of active ingredients was observed during the gastric step (0 to 60 min in simulated gastric fluid), and full-release of the active ingredient was subsequently achieved between 60 min and 180 min, when the oral dosage forms were includes in simulated intestinal fluid (SIF).

### Examples 1-15

### Release Profiles of Oral Dosage Forms Enterically Coated with Protein-Based Films

For Examples 1-15 below, film-forming solutions were applied on small batches of oral dosage forms containing a placebo. Each batch contained between 3600 and 6000 unit oral dosage forms. Unit oral dosage forms were randomly taken and their disintegration into an USP disintegration apparatus was analyzed in simulated gastric fluid (SGF) (USP <701>, 2008). For each batch, 1 capsule was placed in each of the six tubes of the USP disintegration apparatus. Capsules were considered to have passed the test if, after one hour of incubation in SGF, no evidence of disintegration, cracking, or softening was observed on the surface of the coated oral dosage form. Each capsule that passed the test kept their content dry after 60 min of experiment.

Coatings were applied using a pan coating method using the coating parameters as described in Table A.

The oral dosage forms prepared as described in Examples 1-15, as well as the results of their disintegration testing/release profiles, are summarized in Table 1.

### Example 1

Whey protein isolate (WPI) is succinylated at 50% (% of modified NH₂). Film-forming solution composition is set at 4% protein (w/w). Glycerol is used as a plasticizer at a concentration of 1.4% (w/w). No alcohol is added to the film-forming coating solution.

### Example 2

WPI is succinylated at 50% (% of modified NH₂). Film-forming solution composition is set at 10% protein (w/w). A mix of glycerol and triethyl citrate (2:1) is used as a plasticizer at concentration of a concentration of 14% (w/w). Solvent is composed of water and 15% ethanol (w/w).

### Example 3

WPI is succinylated at 50% (% of modified NH₂). Film-forming solution composition is set at 9% protein (w/w). Glycerol is used as a plasticizer at a concentration of 1.5% (w/w). No alcohol is added to the film-forming solution.

### Example 4

WPI is succinylated at 50% (% of modified NH₂). Film-forming solution composition is set at 9% protein (w/w). Glycerol is used as a plasticizer at a concentration of 4% (w/w). Solvent is composed of water and 20% of ethanol (w/w).

### Example 5

WPI is succinylated at 30% (% of modified NH₂). Film-forming solution composition is set at 10% protein (w/w). Glycerol is used as a plasticizer at a concentration of 4% (w/w). No alcohol is added to the film-forming solution.

### Example 6

Capsules were first sub-coated using a hydroxypropyl cellulose (HPC)-based coating. The sub-coating composition is 5.7% (w/w) in polymer. Solvent is composed of water and 47% of ethanol (w/w).

Subsequent to sub-coat application, protein-based enteric coating is applied to capsules. WPI is succinylated at 50% (% of modified NH₂). Film-forming solution composition is set at 9% protein (w/w). Glycerol is used as a plasticizer at a concentration of 3.2% (w/w). Solvent is composed of water and 20% ethanol (w/w).

### Example 7

WPI is succinylated at 50% (% of modified NH₂). Film-forming solution composition is set at 9% protein (w/w). Glycerol is used as a plasticizer at a concentration of 3.2% (w/w). Solvent is composed of water and 20% of ethanol (w/w).

### Example 8

Softgels were first sub-coated using an HPC-based coating. Sub-coating composition is 5.7% (w/w) in polymer. Solvent is composed of water and 47% of ethanol (w/w).

Subsequent to sub-coat application, protein-based enteric coating is applied to the softgels. WPI is succinylated at 50% (% of modified NH₂). Film-forming solution composition is set at 9% protein (w/w). Glycerol is used as a plasticizer at a concentration of 3.2% (w/w). Solvent is composed of water and 20% of ethanol (w/w).

### Example 9

WPI is succinylated at 50% (% of modified NH₂). Film-forming solution composition is set at 10% protein (w/w). Polyethylene glycol 400 (PEG-400) is used as a plasticizer at a concentration of 3% (w/w). Solvent is composed of water and 20% of ethanol (w/w).

### Example 10

WPI is succinylated at 50% (% of modified NH₂). Film-forming solution composition is set at 10% protein (w/w). Propylene glycol (PG) is used as a plasticizer at a concentration of 3% (w/w). Solvent is composed of water and 20% of ethanol (w/w).

### Example 11

Soybean Isolate (SPI) is succinylated at 100% (% of modified NH₂). Film-forming solution composition is set at 9% in protein (w/w). Glycerol is used as a plasticizer at a concentration of 3.2% (w/w). Solvent is composed of water.

### Example 12

SPI is succinylated at 100% (% of modified NH₂). Film-forming solution composition is set at 12% proteins (w/w). PEG-400 is used as a plasticizer at a concentration of 4.8% (w/w). Solvent is composed of water and 20% of ethanol (w/w).

### Example 13

Capsules are first sub-coated using a polyvinyl alcohol (PVA) coating. Sub-coating composition is 22% w/w in polymer. Solvent is composed of water.

Subsequent to sub-coat application, protein-based enteric coating is applied to capsules. SPI is succinylated at 100% (% of modified NH₂). Film-forming solution composition is set at 11% protein (w/w). Glycerol is used as a plasticizer at a concentration of 3.9% (w/w). Solvent is composed of water and 15% of ethanol (w/w).

### Example 14

WPI is succinylated at 50% (% of modified NH₂). Film-forming solution composition is set at 9% in proteins (w/w). Glycerol and PEG-2000 are used as plasticizers at a concentration of 2.8% and 0.4% (w/w), respectively. Solvent is composed of water.

### Example 15

Capsules were first sub-coated using an HPC-based coating. Sub-coating composition is 5.7% in polymer. Solvent is composed of water and 47% of ethanol (w/w).

Subsequent to sub-coat application, protein-based enteric coating is applied to capsules. WPI is succinylated at 50% (% of modified NH₂). Film-forming solution composition is set at 9% protein (w/w). Glycerol is used as a plasticizer at a concentration of 3.5% (w/w). Solvent is composed of water.

TABLE 1

| | | | | | | Number of oral | |
|---|---|---|---|---|---|---|---|
| | | Enteric coating composition (kg) | | | | forms passing | |
| Example | Sub-coat | Protein | Plasticizer(s) | Water | Alcohol | the test | Oral forms |
| 1 | No | 0.4 | 0.142 | 9.858 | — | 5/6* | Capsules “0” |
| | | | | | | | (500 mg) |
| 2 | No | 0.425 | 0.632 | 2.98 | 0.638 | 2/6* | Capsules “0” |
| | | | | | | | (500 mg) |
| 3 | No | 0.45 | 0.081 | 4.919 | — | 6/6 | Capsules “0” |
| | | | | | | | (500 mg) |
| 4 | No | 0.45 | 0.18 | 3.82 | 1 | 6/6 | Softgels |
| | | | | | | | (350 mg) |
| 5 | No | 0.4 | 0.16 | 3.84 | — | 6/6 | Capsules “0” |
| | | | | | | | (500 mg) |
| 6 | HPC 75-150 cP | 0.465 | 0.164 | 4.05 | 1.013 | 6/6 | Capsules “0” |
| | −0.113 kg HPC | | | | | | (500 mg) |
| | −1.05 kg water | | | | | | |
| | −0.937 kg ethanol | | | | | | |
| 7 | No | 0.465 | 0.164 | 4.05 | 1.013 | 6/6 | Capsules “0” |
| | | | | | | | (500 mg) |
| 8 | HPC 75-150 cP | 0.465 | 0.164 | 4.05 | 1.013 | 6/6 | Softgels |
| | −0.113 kg HPC | | | | | | (350 mg) |
| | −1.05 kg water | | | | | | |
| | −0.937 kg ethanol | | | | | | |
| 9 | No | 0.4 | 0.120 | 3.12 | 0.8 | 5/6* | Capsules “0” |
| | | | | | | | (500 mg) |
| 10 | No | 0.4 | 0.120 | 3.08 | 0.8 | 6/6 | Softgels |
| | | | | | | | (350 mg) |
| 11 | No | 0.4 | 0.142 | 4.298 | — | 6/6 | Capsules “0” |
| | | | | | | | (500 mg) |
| 12 | No | 0.45 | 0.18 | 2.82 | 0.75 | 6/6 | Capsules “0” |
| | | | | | | | (500 mg) |
| 13 | PVA | 0.4 | 0.14 | 2.95 | 0.55 | 6/6 | Capsules “0” |
| | −0.193 kg Polymer | | | | | | (500 mg) |
| | −0.879 kg water | | | | | | |
| 14 | No | 0.273 | 0.0956 | 3.008 | — | 6/6 | Capsules “0” |
| | | | | | | | (500 mg) |
| 15 | HPC 75-150 cP | 0.273 | 0.0928 | 3.008 | — | 6/6 | Capsules “0” |
| | −0.072 kg HPC | | | | | | (500 mg) |
| | −0.673 kg water | | | | | | |
| | −0.595 kg ethanol | | | | | | |

*The oral dosage forms with respect to Examples 1, 2 and 9 that failed the disintegration test shown in Table 1 were traced back to capsules that were initially improperly sealed (i.e., the two halves of the capsule were not properly closed). For these initially defective capsules, some oral dosage forms showed cracking evidence on the seal, leading to a wetting of the oral dosage form content, thus resulting in a failure to perfectly respect USP guideline <701>. In Examples 1, 2 and 9, the oral dosage forms whose capsules were initially properly sealed prior to enteric coating successfully passed the disintegration test, thus indicating the need of properly seal the oral dosage forms prior to enteric coating.

### REFERENCES

Abu Diak et al., (2007). The manufacture and characterization of casein films as novel tablet coatings. Trans! Chem E, Part C, Food and bioproducts and processing, 85, 284-290.
Codex Alimentarius (1995). Codex General Standard for Food Additives. Codex STAN 192-1995.
Cunningham & Fegely. (2001). One-step aqueous enteric coating systems: Scale-up evaluation. Pharmaceutical Technology, 36-44.
Dias et al., (2010). Comparative evaluation of enteric film coatings applied in organic solvents. Colorcon Scientific Poster available at:
   http://www.colorcon.com/Iiterature/marketing/mr/Delayed%20Release/Opadry%20Enteric/Chinese/T2219_09138v5_Layout%201.pdf; or at http://www.readbag.com/colorcon-literature-marketing-mr-delayed-release-opadry-enteric-chinese-t2219-09138v5-layout-1.
Food and Drug Administration (FDA). (2006). Database of select committee on Gras substances (SCOGS) reviews.
Gruener and lsmond, (1997), Effects of acetylation and succinylation on the physichochemical properties of the canola 12S globulin. Part I, Food Chemistry, 60, 357-363.
Gueguen et al., (1993). Influence of the dissociation on the surface behaviors of oligomeric seed storage proteins: the example of pea legumin, In: Food Proteins: Structure and Functionality, ed. K. D. Schwenke and R. Mothes, VCH Weinheim (FRG), 281-289
Health Canada (2000). Food and drug regulations. Part B, division 16, table XIII.
Hwang D-C., Damodaran S., (1996), Chemical modification strategies for synthesis of protein-based hydrogel. Journal of Agriculture and Food Chemistry, 44, 751-758.
Li et al., (2010). Aqueous coating dispersion (pseudolatex) of zein improves formulation of sustained-release tablets containing very water-soluble drug. Journal of colloid and interface science, 345, 46-53.
Meghal et al., (2011). Formulation of enteric coated HPMC capsule diclofenac sodium. Research Journal of Pharmaceutical, Biological and Chemical Sciences, 2, 790-797.
Porter et al., (2009). Chapter 33: Development, optimization, and scale-up of process parameters: pan coating. In Y. Qiu, Y. Chen, G. G. Z. Zhang, L. Liu & W. Porter (Eds.), Developing solid oral dosage forms, Pharmaceutical theory and practice (pp. 761-805). New York: Academic Press.
Schwenke Klaus Dieter, Mothes Ralf, Zirwer Dietrich, Gueguen Jacques and Subirade Muriel. 1993. Modification of the structure of 11S globulins from plant seeds by succinylation, In: Food Proteins: Structure and Functionality, ed. K. D. Schwenke and R. Mothes, VCH Weinheim (FRG), 143-153.
Singh et al., (1995), The effect of electrostatic charge interactions on release rates of Gentamicin from collagen matrices, Pharmaceutical research, 12, 1205-1210.
Subirade et al., (1992), Effect of dissociation and conformational changes on the surface behavior of pea legumin, Journal of Colloid and Interface Science, 152, 442-454.
Subirade and Chen, (2008). Food protein derived materials and their use as carriers and delivery systems for active food components, in: Delivery and controlled release of bioactives in foods and nutraceuticals, Woodhead Publishing Ltd, Cambridge. p. 251-271.
The United States Pharmacopoeia—National Formulary (2008). <701> Disintegration. USP Convention Inc., Rockville, Md.
The United States Pharmacopoeia—National Formulary (2008). <711> Dissolution. USP Convention Inc., Rockville, Md.
The United States Pharmacopoeia—National Formulary (2008). <724> Drug Release. USP Convention Inc., Rockville, Md.

1. An enterically coated oral dosage form comprising a core comprising at least one active ingredient, wherein the core is enterically coated with a chemically modified protein, wherein the chemically modified protein comprises:
(a) a chemical modification that causes a decrease in isoelectric point (pI) below that of the corresponding unmodified protein;
(b) a chemical modification that causes a decrease in solubility at acidic pH as compared to that of the corresponding unmodified protein; and/or
(c) a chemical modification that increases the ability of the protein to resist degradation by pepsin when in film-form, as compared to that of the corresponding unmodified protein.

2. (canceled)

3. The enterically coated oral dosage form of claim 1, wherein the decrease in pI is a decrease of 0.1, 0.2, 0.3, 0.4, 0.5, 0.6, 0.7, 0.8, 0.9, 1, 1.5, 2, 2.5, or 3 units, below that of the corresponding unmodified protein.

4. (canceled)

5. (canceled)

6. The enterically coated oral dosage form of claim 1, wherein the chemically modified protein is chemically modified by succinylation, octenyl-succinylation, methylation, acetylation, phosphorylation, acylation, sulfation, carboxylation, arylation, alkylation, deamidation, glutarylation, reaction with an anhydride, ethylenediaminetetraacetic dianhydride (EDTAD), reaction with fluorescein isothiocyanate, reaction with dimethylaminonaphtylsulfonyl chloride (dansyl chloride), or any combination thereof.

7. An enterically coated oral dosage form comprising a core comprising at least one active ingredient, wherein the core is enterically coated with a chemically modified protein wherein the chemically modified protein is chemically modified by succinylation.

8. The enterically coated oral dosage form of claim 1, wherein the chemically modified protein is chemically modified at least at 1%, 5%, 10%, 15%, 20%, 25%, 30%, 35%, 40%, 45%, 50%, 55%, 60%, 65%, 70%, or 75% of its available sites; or between 1% and 100%, 10% and 100%, or 20% and 100% of its available sites.

9. (canceled)

10. The enterically coated oral dosage form of claim 1, wherein the chemically modified protein is a chemically modified water soluble protein.

11. The enterically coated oral dosage form of claim 1, wherein the chemically modified protein comprises at least 1%, at least 1.5%, at least 2%, at least 2.5%, at least 3%, at least 3.5%, at least 4%, at least 4.5%, at least 5%, at least 6%, at least 7%, at least 8%, at least 9%, or at least 10% of lysine residues; or between 1% and 20%, 1% and 15%, 1% and 10%, 1.5% and 20%, 1.5% and 15%, or 1.5% and 10% of lysine residues.

12. (canceled)

13. The enterically coated oral dosage form of claim 1, wherein the chemically modified protein comprises at least 3%, at least 4%, at least 5%, at least 6%, at least 7%, or at least 8% of glutamate and aspartate residues; or between 3% and 20%, 4% and 20%, 5% and 20%, 6% and 20%, 7% and 20%, or 8% and 20% of glutamate and aspartate residues.

14. (canceled)

15. The enterically coated oral dosage form of claim 1, wherein the chemically modified protein is from an animal or plant source, or is from a food protein, a dairy protein, or a leguminous protein.

16-18. (canceled)

19. The enterically coated oral dosage form of claim 15, wherein the chemically modified protein is a chemically modified soy protein, whey protein, pea protein, nut protein, peanut protein, bean protein, lentil protein, wheat globulin protein, corn globulin protein, rice globulin protein, sunflower globulin protein, rape globulin protein, casein protein, animal albumin protein, egg ovalbumin protein, animal collagen protein, or any combination thereof.

20. An enterically coated oral dosage form comprising a core comprising at least one active ingredient, wherein the core is enterically coated with succinylated soy and/or succinylated whey protein, wherein:
(a) less than 20%, less than 15%, less than 10%, or less than 5% of the active ingredient Is released, relative to the initial weight of the active ingredient, upon incubation for 1 hour in simulated gastric fluid (SGF); and/or
(b) at least 20%, 25%, 30%, 35%, 40%, 45%, 50%, 55%, 60%, 65%, 70%, 75%, 80%, 85%, 90%, or 95% of the active ingredient, relative to the initial weight of the active ingredient, is released upon subsequent incubation for 2 hours in simulated intestinal fluid (SIF).

21. The enterically coated oral dosage form of claim 20, wherein the soy protein is from soybean protein extract (SPE) or soybean protein isolate (SPI); or the whey protein is from whey protein extract (WPE), whey protein concentrate (WPC), or whey protein isolate (WPI).

22. (canceled)

23. The enterically coated oral dosage form of claim 1, wherein:
(a) enteric weight gain of the oral dosage form is about 2% to about 30% w/w, about 3% to about 20% w/w; about 5% to about 20% w/w; about 5% to about 18% w/w, or about 8% to about 15% w/w, relative to the initial weight of the oral dosage form; and/or
(b) the enteric coating exhibits swelling of at least 30%, 40%, 50%, 60%, 70%, 80%, 90%, 100%, 110%, or 120% by weight, relative to the initial weight of the enteric coating, within 15 minutes upon incubation in simulated gastric fluid (SGF).

24. (canceled)

25. The enterically coated oral dosage form of claim 1, wherein:
(a) less than 20%, less than 15%, less than 10%, or less than 5% of the active ingredient is released, relative to the initial weight of the active ingredient, upon incubation for 1 hour in simulated gastric fluid (SGF); and/or
(b) at least 20%, 25%, 30%, 35%, 40%, 45%, 50%, 55%, 60%, 65%, 70%, 75%, 80%, 85%, 90%, or 95% of the active ingredient, relative to the initial weight of the active ingredient, is released upon subsequent incubation for 2 hours in simulated intestinal fluid (SIF).

26. (canceled)

27. The enterically coated oral dosage form of claim 1, wherein the enteric coating further comprises:
(i) a plasticizer; and/or
(ii) a sub-coating between the enteric coating and the core.

28. The enterically coated oral dosage form of claim 27, wherein:
(i) the plasticizer is a polyhydric alcohol; an acetate ester; a phthalate ester; a glyceride; an oil; or any combination thereof: and/or
(ii) the sub-coating comprises: a cellulosic based coating, a vinyl based coatings, a glycol, an acrylic coating, and/or a carbohydrate-based coating.

29. The enterically coated oral dosage form of claim 28, wherein:
(i) the plasticizer comprises:
(a) a polyhydric alcohol which is glycerol, propylene glycol, or a combination thereof;
(b) an acetate ester which is glyceryl triacetate, triethyl citrate, or a combination thereof;
(c) a phthalate ester which is diethyl phthalate;
(d) a glyceride is an acetylated monoglyceride; or
(e) an oil which is castor oil, mineral oil, or a combination thereof; and/or
(ii) the sub-coating comprises:
(a) a cellulosic based coating which is: hydroxypropylmethylcellulose, hydroxypropylcellulose, hydroxyethylcellulose, cellulose acetate, or any combination thereof;
(b) a vinyl based coating which is: poly vinyl pyrrolidone, poly vinyl alcohol, poly vinyl pyrrolidone, a poly vinyl acetate copolymer, poly vinyl alcohol, a poly ethylene glycol copolymer, or any combination thereof;
(c) a glycols which is poly ethylene glycol;
(d) an acrylic coating which is an amino alkyl methacrylate copolymer;
(e) a carbohydrate-based coating which is maltodextrins, polydextrose, or a combination thereof; or
(f) any combination of (ii)(a) to (ii)(e).

30-32. (canceled)

33. The enterically coated oral dosage form of claim 1 in the form of an enterically coated tablet, capsule, caplet, softgel, lozenge, pellet, or granule.

34. The enterically coated oral dosage form of claim 1, wherein the active ingredient is a drug, a vitamin, a nutritional supplement, a probiotic, or any combination thereof.

35. The enterically coated oral dosage form of claim 1, wherein the active ingredient is: an antiagreggant, an antiangiogenic, an antiarrythmia, an antibiotic, an antidepressor, an antifungal, an antiviral, an anticholinergic, an antiepileptic, an anticoagulant, an anticonvulsive, an antidiarrhea, an antihistaminic, an antihypertensive, an antiinflammatory, an analgesic, an antalgics, an antipsychotic, an antispasmodic, a synthetic antithyroid, an anxiolytic, a beta-blocker, a cardiotonic, a diuretic, a hypnotic, a hypoglycemic, a hypolipemic, an inhibitor of conversion enzyme, an inhibitor of angiotensin II, an interferon, a mucolytic, a nootropic, a phenylethylamine, a sartan, a triptan, a vitamin, a flavonoid, a peptide, a hormone, an enzyme, a prebiotic, a probiotic, a mineral, an ellagic acid, an omega-3 fatty acid, an omega-6 fatty acid, a terpene, or any combination thereof.

36-46. (canceled)
